(19) United States
(12) Patent Application Publication
(10) Pub. No.: US 2013/0012548 A1
(43) Pub. Date: Jan. 10, 2013

(54) DEUTERIUM-SUBSTITUTED OMEGA-DIPHENYLUREA AND DERIVATIVES THEREOF AND PHARMACEUTICAL COMPOSITIONS COMPRISING THE COMPOUNDS

(75) Inventors: Lidong Xing, Jiangsu (CN); Zelin Sheng, Jiangsu (CN); Guozhang Wu, Jiangsu (CN); Huiping Lu, Jiangsu (CN)

(73) Assignee: SUZHOU ZELGEN BIOPHARMACEUTICAL CO., LTD., Jiangsu (CN)

(21) Appl. No.: 13/635,446

(22) PCT Filed: Mar. 18, 2010
(86) PCT No.: PCT/CN2010/071128

§ 371 Date: Sep. 17, 2012

(51) Int. Cl. A61K 31/44; C07D 209/48; C07D 213/81; A61P 35/00; A61P 35/02; A61P 29/00; A61P 37/00; A61P 13/12; A61P 13/08; C07C 235/46; A61P 9/00

(52) U.S. Cl. 514/346; 546/323; 564/167; 548/480; 546/291

(57) ABSTRACT

This invention relates to deuterated ω-diphenylurea and derivatives and pharmaceutical acceptable salts thereof. And the pharmaceutical compositions comprising the pharmaceutically acceptable carrier and the deuterium-substituted ω-diphenylurea and derivatives and pharmaceutical acceptable salts thereof are also provided. The deuterium-substituted diphenylurea can be used in treatment or prevention of cancer and other related diseases.

### FIELD OF INVENTION

The invention relates to deuterated ω-diphenylurea and derivatives thereof and pharmaceutical compositions comprising the compounds.

### BACKGROUND OF INVENTION

The ω-diphenylurea derivatives are known as the compounds with c-RAF kinase inhibition activity. For example, WO2000/042012 disclosed a class of ω-carboxyl-aryl-substituted diphenylurea and the use thereof for treating cancer and related diseases.

Initially, ω-diphenylurea compounds, such as Sorafenib, were firstly found as the inhibitors of c-RAF kinase. The other studies had shown that they could also inhibit the MEK and ERK signal transduction pathways and activities of tyrosine kinases including vascular endothelial growth factor receptor-2 (VEGFR-2), vascular endothelial growth factor receptor-3 (VEGFR-3), and platelet-derived growth factor receptor-β (PDGFR-β) (Curr Pharm Des 2002, 8, 2255-2257). Therefore, they were called multi-kinase inhibitors that resulted in dual anti-tumor effects.

Sorafenib (trade name Nexavar), a novel oral multi-kinase inhibitor, was developed by Bayer and Onyx. In December 2005, based on its outstanding performance in phase III clinical trials for treating advanced renal cell carcinoma, Sorafenib was approved by FDA for treating advanced renal cell carcinoma. It was marketed in China in November 2006. However, Sorafenib has various side-effects, such as hypertension, weight loss, rash and so on.

Therefore, novel compounds with raf kinase inhibition activity or better pharmacodynamic properties are still needed to be developed.

### SUMMARY OF INVENTION

The object of the invention is to provide novel compounds with raf kinase inhibition activity and better pharmacodynamic properties and the uses thereof.

In the first aspect, the invention provides a deuterium-substituted ω-diphenylurea compound of formula (I), or the crystal forms, pharmaceutically acceptable salts, hydrates or solvates thereof:

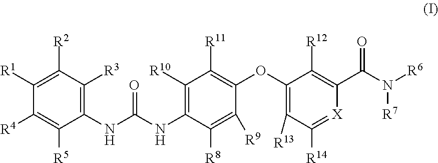

wherein,

X is N or N⁺-O⁻;

R¹ is halogen (such as F, Cl or Br), one or more deuterium-substituted or perdeuterated C1-C4 alkyl;

R² is non-deuterated C1-C4 alkyl, one or more deuterium-substituted or perdeuterated C1-C4 alkyl, or partly or totally halogen-substituted C1-C4 alkyl;

each of R³, R⁴, R⁵, R⁸, R⁹, R¹⁰, R¹¹, R¹², R¹³, and R¹⁴ is independently hydrogen, deuterium, or halogen (such as F, Cl or Br);

R⁶ is hydrogen, deuterium, or one or more deuterium-substituted or perdeuterated C1-C4 alkyl;

R⁷ is hydrogen, deuterium, or one or more deuterium-substituted or perdeuterated C1-C4 alkyl; and provided that at least one of R², R³, R⁴, R⁵, R⁶, R⁷, R⁸, R⁹, R¹⁰, R¹¹, R¹², R¹³ or R¹⁴ is deuterated or is deuterium.

In one embodiment, the deuterium content in a deuterium-substituted position is at least greater than the natural abundance of deuterium (0.015%), preferably >30%, more preferably >50%, more preferably >75%, or more preferably >95%, or more preferably >99%.

In one embodiment, compounds of formula (I) contain at least one deuterium atom, preferably three deuterium atoms, and more preferably five deuterium atoms.

In one embodiment, R¹ is halogen, preferably chlorine.

In one embodiment, R² is trifluoromethyl.

In one embodiment, R⁶ or R⁷ is independently selected from hydrogen, deuterium, deuterated methyl, or deuterated ethyl; preferably, mono-deuterated methyl, bi-deuterated methyl, tri-deuterated methyl, mono-deuterated ethyl, bi-deuterated ethyl, tri-deuterated ethyl, tetra-deuterated ethyl, or penta-deuterated ethyl.

In one embodiment, R⁶ or R⁷ is independently selected from hydrogen, methyl or tri-deuterated methyl.

In one embodiment, R³, R⁴ or R⁵ is independently selected from hydrogen or deuterium.

In one embodiment, R⁸, R⁹, R¹⁰ or R¹¹ is independently selected from hydrogen or deuterium.

In one embodiment, R¹², R¹³ or R¹⁴ is independently selected from hydrogen or deuterium.

In one embodiment, said compounds are selected from:

N-(4-chloro-3-(trifluoromethyl)phenyl)-N′-(4-(2-(N-(methyl-d₃)aminoformyl)-4-pyridyloxy)phenyl)urea (or 4-(4-(3-(4-chloro-3-(trifluoromethyl)phenyl)ureido)phenoxy)-N-(methyl-d₃)picolinamide);

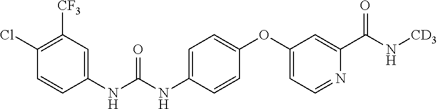 [SMILES: [2H]C([2H])([2H])NC(=O)c1cc(Oc2ccc(NC(=O)Nc3ccc(Cl)c(C(F)(F)F)c3)cc2)ccn1]

4-(4-(3-(4-chloro-3-(trifluoromethyl)phenyl)ureido)phenoxy)-2-(N-(methyl-d₃)aminoformyl)pyridine-1-oxide;

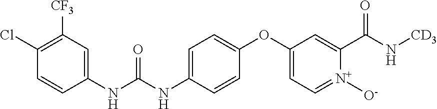 [SMILES: [2H]C([2H])([2H])NC(=O)c1cc(Oc2ccc(NC(=O)Nc3ccc(Cl)c(C(F)(F)F)c3)cc2)cc[n+]1[O-]]

N-(4-chloro-3-(trifluoromethyl)phenyl)-N′-(4-(2-(N-(methyl-d₂)aminoformyl)-4-pyridyloxy)phenyl)urea;

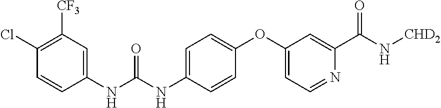

N-(4-chloro-3-(trifluoromethyl)phenyl)-N′-(4-(2-(N-(methyl-d)aminoformyl)-4-pyridyloxy)phenyl)urea;

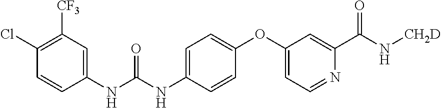

N-(4-chloro-3-(methyl-d₃)phenyl)-N′-(4-(2-(N-methylaminoformyl)-4-pyridyloxy)phenyl)urea;

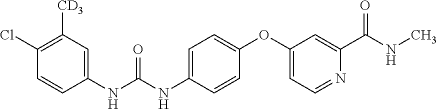 [SMILES: [2H]C([2H])([2H])c1cc(NC(=O)Nc2ccc(Oc3ccnc(C(=O)NC)c3)cc2)ccc1Cl]

N-(4-chloro-3-(trifluoromethyl)phenyl)-N′-(4-(2-d-6-(N-methylaminoformyl)-4-pyridyloxy)phenyl)urea;

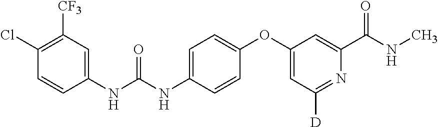 [SMILES: [2H]c1cc(Oc2ccc(NC(=O)Nc3ccc(Cl)c(C(F)(F)F)c3)cc2)cc(C(=O)NC)n1]

N-(4-chloro-3-(trifluoromethyl)phenyl)-N′-(2-d-4-(2-d-6-(N-methylaminoformyl)-4-pyridyloxy)phenyl)urea;

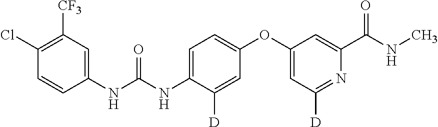 [SMILES: [2H]c1cc(Oc2ccc(NC(=O)Nc3ccc(Cl)c(C(F)(F)F)c3)c([2H])c2)cc(C(=O)NC)n1]

N-(4-chloro-3-(trifluoromethyl)phenyl)-N′-(2,6-d₂-4-(2-d-6-(N-methylaminoformyl)-4-pyridyloxy)phenyl)urea;

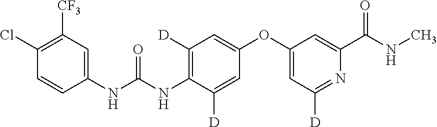 [SMILES: [2H]c1cc(Oc2cc([2H])c(NC(=O)Nc3ccc(Cl)c(C(F)(F)F)c3)c([2H])c2)cc(C(=O)NC)n1]

N-(4-chloro-3-(methyl-d₃)phenyl)-N′-(4-(2-d-6-(N-methylaminoformyl)-4-pyridyloxy)phenyl)urea;

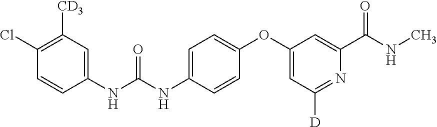 [SMILES: [2H]c1cc(Oc2ccc(NC(=O)Nc3ccc(Cl)c(C([2H])([2H])[2H])c3)cc2)cc(C(=O)NC)n1]

N-(4-chloro-3-(trifluoromethyl)phenyl)-N′-(4-(2-(N-methyl-(methyl-d₃)aminoformyl)-4-pyridyloxy)phenyl)urea;

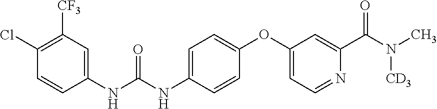 [SMILES: [2H]C([2H])([2H])N(C)C(=O)c1cc(Oc2ccc(NC(=O)Nc3ccc(Cl)c(C(F)(F)F)c3)cc2)ccn1]

N-(4-chloro-3-(trifluoromethyl)phenyl)-N-(4-(2-(N,N-di(methyl-d₃)aminoformyl)-4-pyridyloxy)phenyl)urea;

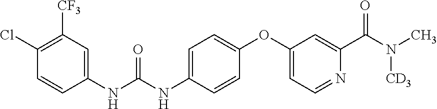 [SMILES: [2H]C([2H])([2H])N(C)C(=O)c1cc(Oc2ccc(NC(=O)Nc3ccc(Cl)c(C(F)(F)F)c3)cc2)ccn1]

N-(4-chloro-3-(trifluoromethyl)phenyl)-N′-(2,6-d₂-4-(2-(N-(methyl-d₃)aminoformyl)-4-pyridyloxy)phenyl)urea;

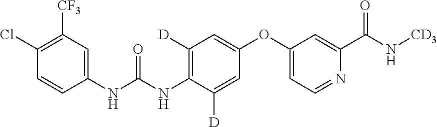 [SMILES: [2H]c1cc(Oc2ccnc(C(=O)NC([2H])([2H])[2H])c2)cc([2H])c1NC(=O)Nc1ccc(Cl)c(C(F)(F)F)c1]

N-(4-chloro-3-(trifluoromethyl)phenyl)-N′-(4-(2-d-6-(N-(methyl-d₃)aminoformyl)-4-pyridyloxy)phenyl)urea;

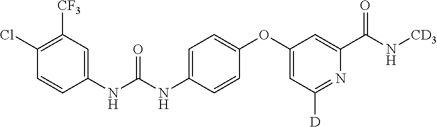 [SMILES: [2H]c1cc(Oc2ccc(NC(=O)Nc3ccc(Cl)c(C(F)(F)F)c3)cc2)cc(C(=O)NC([2H])([2H])[2H])n1]

N-(4-chloro-3-(trifluoromethyl)phenyl)-N′-(4-(2-(N-(ethyl-1,1-d₂)aminoformyl)-4-pyridyloxy)phenyl)urea;

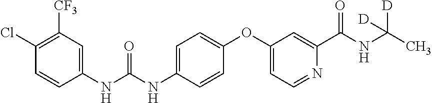 [SMILES: [2H]C([2H])(C)NC(=O)c1cc(Oc2ccc(NC(=O)Nc3ccc(Cl)c(C(F)(F)F)c3)cc2)ccn1]

N-(4-chloro-3-(trifluoromethyl)phenyl)-N′-(4-(2-(N-(ethyl-d₅)aminoformyl)-4-pyridyloxy)phenyl)urea;

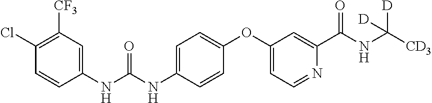 [SMILES: [2H]C([2H])([2H])C([2H])([2H])NC(=O)c1cc(Oc2ccc(NC(=O)Nc3ccc(Cl)c(C(F)(F)F)c3)cc2)ccn1]

or

N-(4-chloro-3-(methyl-d₃)phenyl)-N′-(4-(2-(N-(methyl-d₃)aminoformyl)-4-pyridyloxy)phenyl)urea.

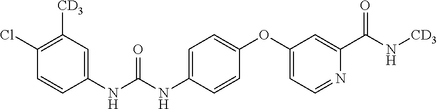 [SMILES: [2H]C([2H])([2H])NC(=O)c1cc(Oc2ccc(NC(=O)Nc3ccc(Cl)c(C([2H])([2H])[2H])c3)cc2)ccn1]

In the second aspect, the invention provides a method for preparing a pharmaceutical composition, comprising: mixing a pharmaceutically acceptable carrier with the compounds according to the first aspect of the invention, or the crystal forms, pharmaceutically acceptable salts, hydrates or solvates thereof, thereby forming the pharmaceutical composition.

In the third aspect, the invention provides a pharmaceutical composition, comprising a pharmaceutically acceptable carrier and the compounds according to the first aspect of the invention, or the crystal forms, pharmaceutically acceptable salts, hydrates or solvates thereof.

In one embodiment, said pharmaceutical composition comprises the injection, capsule, tablet, pill, powder, or granule.

In one embodiment, said pharmaceutical composition further comprises additional medicaments, which are the medicaments for treating cancer, cardiovascular diseases, inflammation, immune diseases, nephrosis, angiogenesis, or prostatosis.

Preferably, said medicaments include but are not limited to: 5-fluorouracil, AV412, avastin™ (bevacizumab), bexarotene, bortezomib, calcitriol, canertinib, capecitabine, carboplatin, celecoxib, cetuximab, CHR-2797, cisplatin, dasatinib, digoxin, enzastaurin, Erlotinib, etoposide, everolimus, fulvestrant, gefitinib, gemcitabine, genistein, imatinib, irinotecan, lapatinib, lenalidomide, letrozole, leucovorin, matuzumab, oxaliplatin, paclitaxel, panitumumab, pegfilgrastin, peglated α-interferon, pemetrexed, Polyphenon® E, satraplatin, sirolimus, sutent (sunitinib), sulindac, taxotere, temodar (temozomolomide), Torisel (temsirolimus), TG01, tipifarnib, trastuzumab, valproic acid, vinflunine, Volociximab, vorinostat and XL647.

In the fourth aspect, the invention provides the use of the compounds according to the first aspect of the invention, or the crystal forms, pharmaceutically acceptable salts, hydrates or solvates thereof for preparing a pharmaceutical composition that inhibits phosphokinases (such as raf kinase).

In one embodiment, said pharmaceutical composition is used for treating and preventing the following diseases: cancer, cardiovascular diseases, inflammation, immune diseases, nephrosis, angiogenesis, or prostatosis.

In one embodiment, said cancer includes (but is not limited to): non-small-cell lung cancer, uterine cancer, rectal cancer, cerebral cancer, head cancer, neck cancer, bladder cancer, prostate cancer, breast cancer, solid tumor, kidney cancer, leukaemia, liver cancer, gastric cancer, or pancreatic cancer.

In the fifth aspect, the invention provides a method of treatment, comprising a step of administrating said compound according to the first aspect of the invention, or the crystal forms, pharmaceutically acceptable salts, hydrates or solvates thereof, or said pharmaceutical composition according to the third aspect of the invention to a subject in need of, thereby inhibiting phosphokinases (such as raf kinase). Preferably, said disease includes cancer, cardiovascular diseases, inflammation, immune diseases, nephrosis, angiogenesis, or prostatosis.

In the sixth aspect, the invention provides a method for preparing N-(4-chloro-3-(trifluoromethyl)phenyl)-N′-(4-(2-(N-(m ethyl-d₃)am inoformyl)-4-pyridyloxy)phenyl)urea,

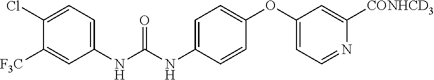

which comprises:

(a) in an inert solvent and in the presence of a base, reacting compound III with compound V, thereby forming said compound, wherein X is Cl, Br, or I;

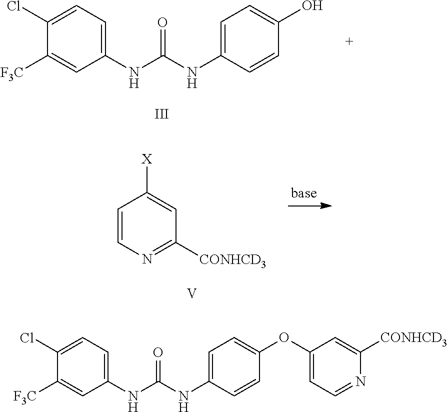

or comprises:

(b) in an inert solvent, reacting compound IX with CD₃NH₂ or CD₃NH₂.HCl, thereby forming said compound, wherein R is C1-C8 straight-chain or branched chain alkyl, or aryl;

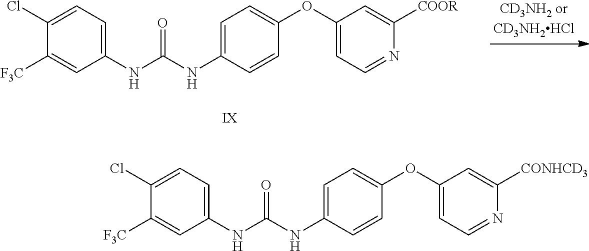

or comprises:

(c) in an inert solvent, reacting 1-chloro-4-isocyanato-2-(trifluoromethyl)benzene (VIII) with compound 5, thereby forming said compound;

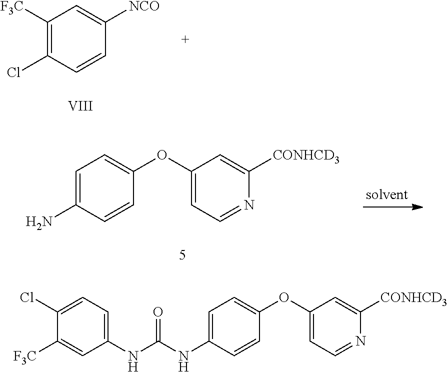

or comprises:

(d) in an inert solvent and in the presence of CDI and CH₂Cl₂, reacting compound 5 with compound 6, thereby forming said compound.

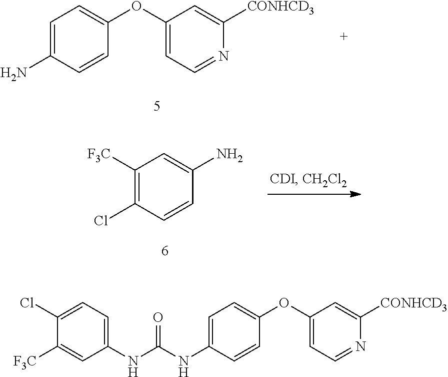

In one embodiment, compound III is prepared as follows:

(i) condensing p-hydroxyaniline (I) and 4-chloro-3-trifluoromethylaniline (II), thereby forming compound III:

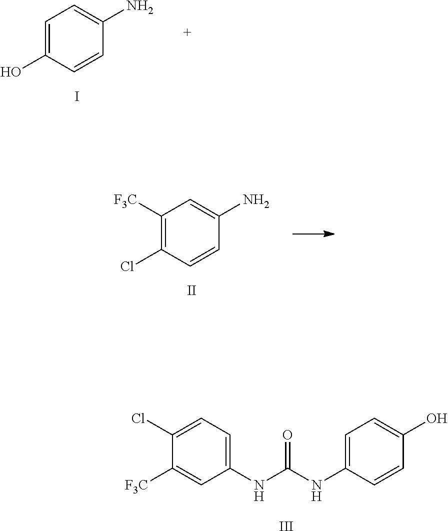

or (ii) reacting p-methoxyaniline (X) with 4-chloro-3-trifluoromethylaniline (II) or 1-chloro-4-isocyanato-2-(trifluoromethyl)benzene (VIII), thereby forming compound XI:

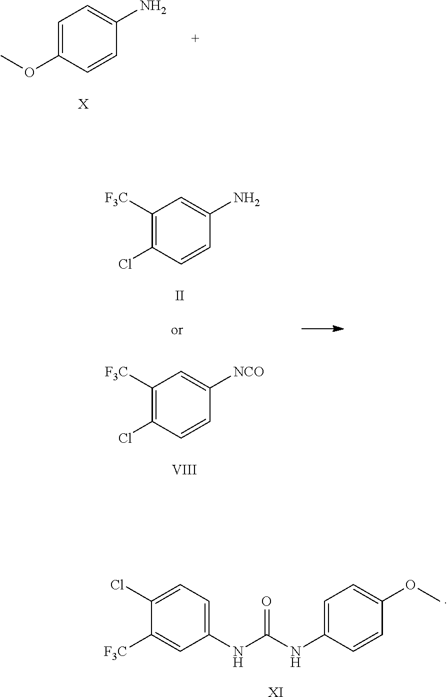

and, in the presence of an acid or a base, demethylating compound XI to give compound III.

In one embodiment, compound VII is prepared as follows:

In the presence of a base, reacting compound VI with p-hydroxyaniline, thereby forming compound VII,

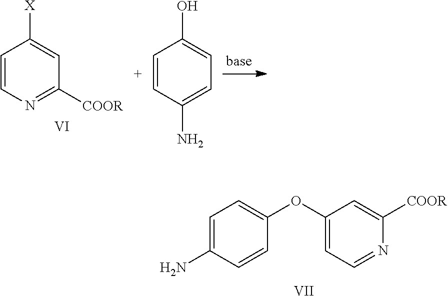

wherein X is fluorine, chlorine, or iodine; R is straight-chain or branched chain C1-C8 alkyl, or aryl.

It should be understood that in the present invention, any of the technical features specifically described above and below (such as in the Examples) can be combined with each other, thereby constituting new or preferred technical solutions that are not described one by one in the specification.

### DESCRIPTION OF FIGURES

FIG. 1 shows the curves of drug concentration (ng/ml) in plasma after oral administration of 3 mg/kg of the control compound CM4306 to the male SD rats.

FIG. 2 shows the curves of drug concentration (ng/ml) in plasma after oral administration of 3 mg/kg of the compound CM4307 to the male SD rats.

FIG. 3 shows the curves of inhibition efficacy of CM4306 and CM4307 in nude mice xenograft model inoculated with human liver cell cancer cell SMMC-7721. In this figure, “treatment” means that the treating period was 14 days, followed by the observation period after administration was stopped. The five days before treatment was the period for preparing animal models.

### DETAILED DESCRIPTION OF INVENTION

After studies, the inventors unexpectedly discovered that, compared with the un-deuterated compound, the deuterated ω-diphenylurea of the invention and the pharmaceutically acceptable salts thereof possessed better pharmacokinetic and/or pharmacodynamic properties. Therefore, they were more suitable as raf kinase inhibitors for preparing medicaments to treat cancer and the relevant diseases. Based on this discovery, the inventors completed the present invention.

Taking the deuterated compound CM4307 (chemical name, N-(4-chloro-3-(trifluoromethyl)phenyl)-N′-(4-(2-(N-(methyl-d₃)aminoformyl)-4-pyridyloxy)phenyl)urea) and the un-deuterated compound CM4306 (chemical name, N-(4-chloro-3-(trifluoromethyl)phenyl)-N′-(4-(2-(N-methylaminoformyl)-4-pyridyloxy)phenyl)urea) as an example,

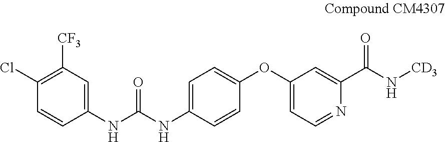 [SMILES: [2H]C([2H])([2H])NC(=O)c1cc(Oc2ccc(NC(=O)Nc3ccc(Cl)c(C(F)(F)F)c3)cc2)ccn1]

the results of pharmacokinetic test showed that the half life (T_{1/2}) of CM4307 was longer, the AUC_{0-∞} of CM4307 increased significantly and the apparent clearance of CM4307 decreased compared to CM4306.

The results of pharmacodynamic test performed in the nude mouse model inoculated with human liver cancer cell SMMC-7721 showed that, after intragastric administration at 100 mg/kg per day for two weeks, the relative tumor increment rate T/C (%) as an evaluation index of CM4306 anti-tumor activity was 32.2%, while that of CM4307 was 19.6%. Therefore, the absolute value of anti-tumor activity increased over 10%, the relative value increased about 60% (32.2%/19.6%−1=64%), and CM4307 showed more notable tumor-inhibiting effect.

Definition

As used herein, the term “halogen” refers to F, Cl, Br and I. Preferably, halogen is selected from F, Cl, and Br.

As used herein, the term “alkyl” refers to straight-chain or branched chain alkyl. Preferable alkyl is C1-C4 alkyl, such as methyl, ethyl, propyl, iso-propyl, butyl, iso-butyl, tert-butyl etc.

As used herein, the term “deuterated” means that one or more hydrogens of compounds or groups are substituted by deuterium. “Deuterated” can be mono-substituted, bi-substituted, multi-substituted or total-substituted. The terms “one or more deuterium-substituted” and “substituted by deuterium once or more times” can be used interchangeably.

In one embodiment, the deuterium content in a deuterium-substituted position is at least greater than the natural abundance of deuterium (0.015%), preferably >50%, more preferably >75%, more preferably >95%, more preferably >97%, more preferably >99%, more preferably >99.5%.

In one embodiment, the compound of formula (I) comprises at least one deuterium atom, preferably 3 deuterium atoms, and more preferably 5 deuterium atoms.

Active Ingredients

As used herein, the term “compound of the invention” refers to the compounds of formula (I). This term also includes various crystal forms, pharmaceutically acceptable salts, hydrates or solvates of the compounds of formula (I).

As used herein, the term “pharmaceutically acceptable salts” refers to the salts which are suitable for medicine and formed by the compounds of the invention and acid or base. Pharmaceutically acceptable salts include inorganic salts and organic salts. A preferred salt is formed by the compound of the invention and acid. The acid suitable for forming salts includes, but not limited to, inorganic acid, such as hydrochloric acid, hydrobromic acid, hydrofluoric acid, sulfuric acid, nitric acid, phosphoric acid; organic acid, such as formic acid, acetic acid, propionic acid, oxalic acid, malonic acid, succinic acid, fumaric acid, maleic acid, lactic acid, malic acid, tartaric acid, citric acid, picric acid, methanesulfonic acid, benzene methanesulfonic acid, benzene sulfonic acid; and acidic amino acid, such as aspartic acid, glutamic acid.

Preparation

The preparation methods of compounds (I) are described in detail as below. However, these specific methods are not provided for the limitation of the invention. The compounds of the invention can be readily prepared by optionally combining any of the various methods described in the specification or with various methods known in the art, and such combination can easily be carried out by the skilled in the art.

The method for preparing un-deuterated w-diphenylurea and the physiologically compatible salts thereof used in the invention is known. The deuterated w-diphenylurea can be prepared in the same route using the corresponding deuterated compounds as the starting materials. For example, compound (I) can be prepared according to the method described in WO2000/042012, except that the deuterated material is used in the reaction instead of un-deuterated material.

In general, during the preparation, each reaction is conducted in an inert solvent, at a temperature between room temperature to reflux temperature (such as 0-80° C., preferably 0-50° C.). Generally, the reaction time is 0.1-60 hours, preferably, 0.5-48 hours.

Taking CM4307 as an example, an optimized preparation route is shown as follows:

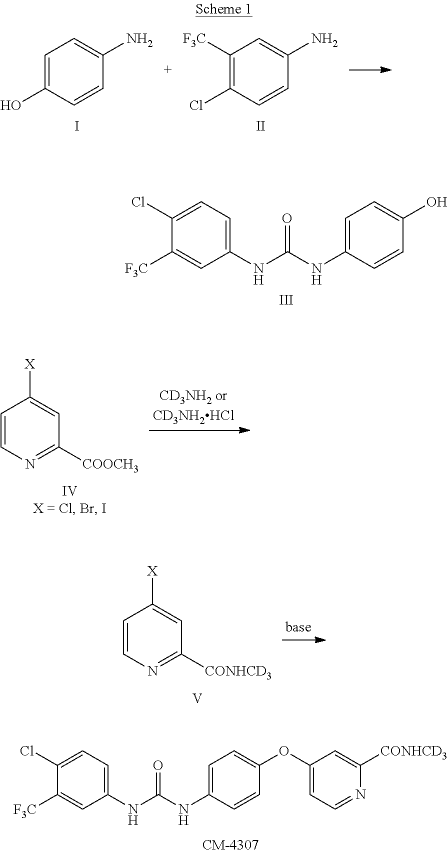

As shown in Scheme 1, in the presence of N,N′-carbonyldiimidazole, phosgene or triphosgene, 4-aminophenol (Compound I) reacts with 3-trifluoromethyl-4-chloro-aniline (Compound II) to give 1-(4-chloro-3-(trifluromethyl)phenyl)-3-(4-hydroxyphenyl)urea (Compound III). 2-(N-(methyl-d₃)) carbamoyl pyridine (Compound V) is obtained by reacting methyl picolinate (Compound IV) with (methyl-d₃)amine or (methyl-d₃)amine hydrochloride directly or in the presence of the base such as sodium carbonate, potassium carbonate, sodium hydroxide, triethylamine, pyridine and the like. In the presence of base (such as potassium tert-butoxide, sodium hydride, potassium hydride, potassium carbonate, cesium carbonate, potassium phosphate, potassium hydroxide, sodium hydroxide) and an optional catalyst (such as cuprous iodide and proline, or cuprous iodide and picolinic acid), Compound III reacts with Compound V to form compound CM-4307. The above reactions are conducted in an inert solvent, such as dichloromethane, dichloroethane, acetonitrile, n-hexane, toluene, tetrahydrofuran, N,N-dimethylformamide, dimethyl sulfoxide and so on, and at a temperature of 0-200° C.

Another preferred process for preparing CM4307 is shown as below:

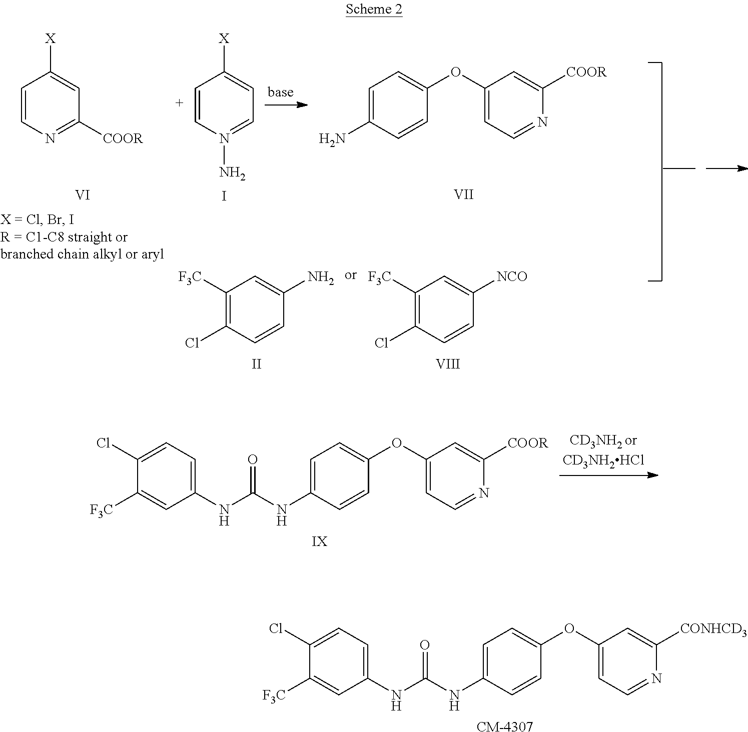

As shown in Scheme 2, amine (Compound VII) is obtained by reacting methyl picolinate (Compound VI) with 4-aminophenol (Compound I) in the presence of base (such as potassium tert-butoxide, sodium hydride, potassium hydride, potassium carbonate, cesium carbonate, potassium phosphate, potassium hydroxide, sodium hydroxide) and an optional catalyst (such as cuprous iodide and proline, or cuprous iodide and pyridine carboxylic acid). The urea (Compound IX) is obtained by reacting Compound VII with Compound II in the presence of N,N′-carbonyldiimidazole, phosgene or triphosgene, or with 1-chloro-4-isocyanato-2-(trifluoromethyl)benzene (Compound VIII). Compound CM4307 is obtained by reacting Compound IX with (methyl-d₃)amine or (methyl-d₃)amine hydrochloride directly, or in the presence of base (such as sodium carbonate, potassium carbonate, sodium hydroxide, triethylamine, pyridine and the like). The above reactions are conducted in an inert solvent, such as dichloromethane, dichloroethane, acetonitrile, n-hexane, toluene, tetrahydrofuran, N,N-dimethylformamide, dimethyl sulfoxide and so on, and at a temperature of 0-200° C.

Another preferred process for preparing CM4307 is shown as below:

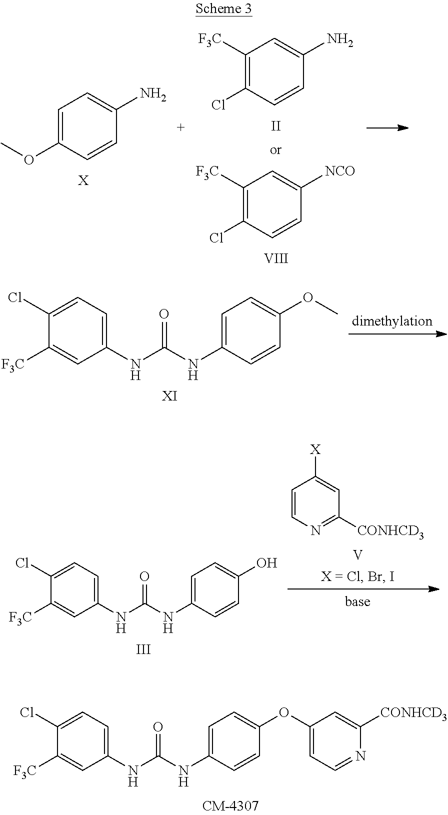

As shown in Scheme 3, the urea (Compound XI) is obtained by reacting 4-methyloxyphenylamine (Compound X) with Compound II in the presence of N,N′-carbonyldiimidazole, phosgene or triphosgene, or with 1-chloro-4-isocyanato-2-(trifluoromethyl)benzene (Compound VIII). 1-(4-chloro-3-(trifluromethyl)phenyl)-3-(4-hydroxyphenyl)urea (Compound III) is obtained using any of demethylation methods known in the art. Compound CM4307 is obtained by reacting Compound III with Compound V by the same method as described in Scheme 1, or any methods known in the art. The above reactions are conducted in an insert solvent, such as dichloromethane, dichloroethane, acetonitrile, n-hexane, toluene, tetrahydrofuran, N,N-dimethylformamide, dimethyl sulfoxide and so on, and at a temperature of 0-200° C.

Another particularly preferred process for preparing CM4307 is shown as below:

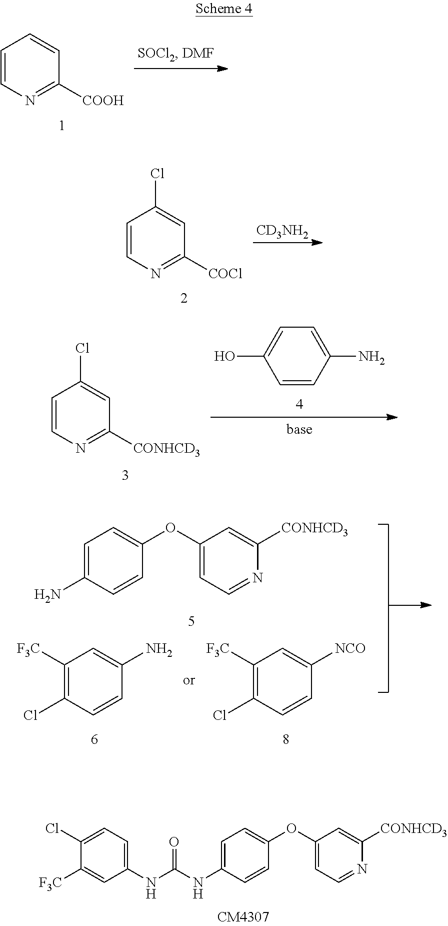

The deuterium can be introduced by using deuterated methylamine. Deuterated methylamine can be prepared using a method known in the art as below, for example, hydrogenation of deuterated nitromethane shown as follows:

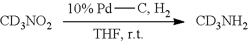

wherein r.t. means room temperature.

Alternatively, deuterated methylamine or the hydrochloride thereof can be prepared through the following reactions. Deuterated nitromethane is obtained by reacting nitromethane with deuterium water in the presence of base (such as sodium hydride, potassium hydride, deuterated sodium hydroxide, deuterated potassium hydroxide, potassium carbonate and the like) or phase-transfer catalyst. The above experiment can be repeated if necessary, to produce deuterated nitromethane in high purity. Deuterated nitromethane is reduced in the presence of zinc powder, magnesium powder, iron, or nickel and the like to form deuterated methylamine or the hydrochloride thereof.

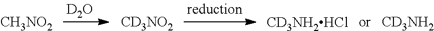

Furthermore, deuterated methylamine or the hydrochloride thereof can be obtained through the following reactions.

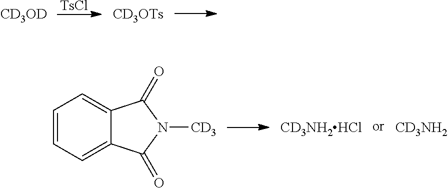

The key intermediate 3 can be synthesized from deuterium methanol (CD₃OD) through the following reactions.

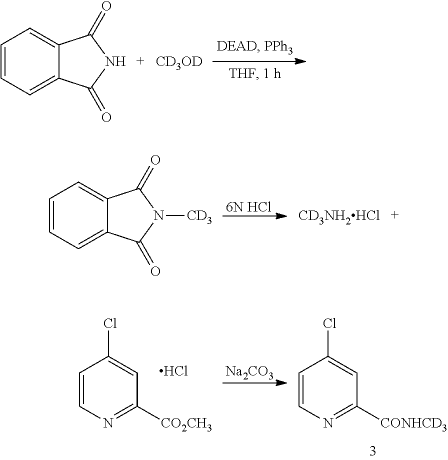

The detailed preparation procedure is described in Example 1.

Pharmaceutical Composition and the Administration Thereof

The compounds of the invention possess outstanding activity of inhibiting kinases, such as raf kinases. Therefore, the compounds of the invention and the crystal forms, the pharmaceutically acceptable inorganic or organic salts, hydrates or solvates thereof, and the pharmaceutical compositions comprising compounds of the invention as a main active ingredient, can be used for treating, preventing and alleviating diseases mediated by kinases (e.g. raf kinase). Based on the prior art, the compounds of the invention can treat the following diseases: cancer, cardiovascular diseases, obesity, diabetes etc.

Pharmaceutical composition of the invention comprises the compounds of the invention or the pharmaceutical acceptable salts thereof in safe and effective dosage range and pharmaceutically acceptable excipients or carriers. The term “safe and effective dosage” refers to the amount of the compounds which is enough to improve the patient's condition without any serious side effect. Generally, the pharmaceutical composition contains 1-2000 mg compounds of the invention per dose. Preferably, 10-200 mg compounds of the invention per dose. Preferably, “per dose” means one capsule or tablet.

“Pharmaceutically acceptable carrier” means one or more compatible solid or liquid fillers or gel materials, which are suitable for human, and must have sufficient purity and sufficiently low toxicity. “Compatibility” herein means that the components of the compositions can be blended with the compounds of the invention or with each other, and would not significantly reduce the efficacy of the compounds. Some examples of pharmaceutically acceptable carriers include cellulose and the derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agents, flavoring agents, stabilizers, antioxidants, preservatives, pyrogen-free water, etc.

There is no special limitation of administration mode for the compounds or pharmaceutical compositions of the invention, and the representative administration mode includes (but is not limited to): oral, intratumoral, rectal, parenteral (intravenous, intramuscular or subcutaneous), and topical administration.

Solid dosage forms for oral administration include capsules, tablets, pills, powders and granules. In these solid dosage forms, the active compounds are mixed with at least one conventional inert excipient (or carrier), such as sodium citrate or CaHPO₄, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as, glycerol; (d) disintegrating agents such as agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, stearin calcium, magnesium stearate, solid polyethylene glycol, sodium lauryl sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain a opaque agent. The release of the active compounds or compounds in the compositions can be released in a delayed mode in a given portion of the digestive tract. Examples of the embedding components include polymers and waxes. If necessary, the active compounds and one or more above excipients can form microcapsules.

Liquid dosage forms for oral administration include pharmaceutically acceptable emulsions, solutions, suspensions, syrups or tinctures. In addition to the active compounds, the liquid dosage forms may contain any conventional inert diluents known in the art such as water or other solvents, solubilizers and emulsifiers, for example, ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combination thereof.

Besides these inert diluents, the composition may also contain additives such as wetting agents, emulsifiers, and suspending agent, sweetener, flavoring agents and perfume.

In addition to the active compounds, the suspension may contain suspending agent, for example, ethoxylated isooctadecanol, polyoxyethylene sorbitol and sorbitan esters, microcrystalline cellulose, methanol aluminum and agar, or the combination thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms for topical administration of compounds of the invention include ointments, powders, patches, aerosol, and inhalants. The active ingredients are mixed with physiologically acceptable carriers and any preservatives, buffers, or propellant if necessary, under sterile conditions.

The compounds of the invention can be administrated alone, or in combination with any other pharmaceutically acceptable compounds.

When the pharmaceutical compositions are used, a safe and effective amount of compound of the present invention is applied to a mammal (such as human) in need of, wherein the dose of administration is a pharmaceutically effective dose. For a person weighed 60 kg, the daily dose is usually 1-2000 mg, preferably 20-500 mg. Of course, the particular dose should also depend on various factors, such as the route of administration, patient healthy status, which are well within the skills of an experienced physician.

Compared with the un-deuterated compounds known in the art, the compounds of the invention possess lots of advantages. The main advantages of the present invention include:

(1) Compounds of the present invention possess excellent activities of inhibiting phosphokinases such as raf kinases.

(2) The metabolism in the organism is altered through deuteration, so that the metabolism of drugs becomes more difficult in vivo, which reduces the first-pass effect. In such cases, the dose can be changed and long-acting preparations can be formed. Further the applicability can be improved by using long-acting preparations.

(3) The pharmacokinetics is also changed through deuteration. Since another hydrate film is fully formed by deuterated compounds, the distribution of deuterated compounds in organisms is significantly different from that of the non-deuterated compounds.

(4) Hydrogen atoms in the compounds are replaced by deuterium. Therefore, the concentration of the parent compound in animals can be increased because of the isotope effect, thereby improving drug efficacy.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacture's instructions. Unless indicated otherwise, parts and percentage are calculated by weight.

### EXAMPLE 1

### Preparation of N-(4-ehloro-3-(trifluoromethyl)phenyl)-N′-(4-(2-(N-(methyl-d₃)aminoformyl)-4-pyridyloxy)phenyl)urea (Compound CM4307)

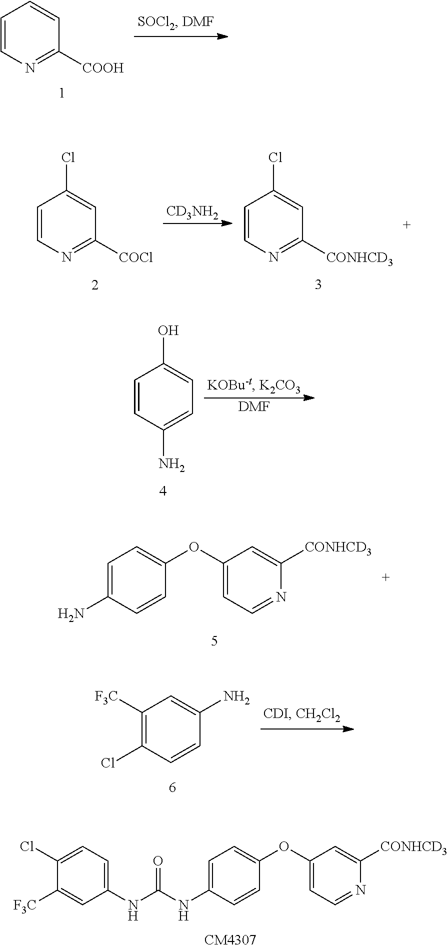

1. Preparation of 4-chloropyridine-2-(N-(methyl-d₃))carboxamide (3)

Into a 250 mL single-neck round-bottom flask equipped with waste gas treatment equipments, thionyl chloride (60 mL) was added. Anhydrous DMF (2 mL) was dropwise added slowly while keeping temperature at 4050° C. After addition, the mixture was stirred for 10 min, and then nicotinic acid (20 g, 162.6 mmol) was added in portions in 20 min. The color of the solution gradually changed from green into light purple. The reaction mixture was heated to 72° C., and refluxed for 16 hours with agitation. A great amount of solid precipitate formed. The mixture was cooled to room temperature, diluted with toluene (100 mL) and concentrated to almost dry. The residue was diluted with toluene again and concentrated to dry. The residue was filtered and washed with toluene to give 4-chloro-pyridine-2-carbonyl chloride as a light yellow solid. The solid was slowly added into a saturated solution of (methyl-d₃)amine in tetrahydrofuran in an ice-bath. The mixture was kept below 5° C. and stirred for 5 hours. Then, the mixture was concentrated and ethyl acetate was added to give a white solid precipitate. The mixture was filtered, and the filtrate was washed with saturated brine, dried over sodium sulfate and concentrated to give 4-chloropyridine-2-(N-(methyl-d₃)) carboxamide (3) (20.68 g, 73% yield) as a light yellow solid.

¹H NMR (CDCl₃, 300 MHz): 8.37 (d, 1H), 8.13 (s, 1H), 7.96 (br, 1H), 7.37 (d, 1H).

2. Preparation of 4-(4-aminophenoxy)-2-pyridine-(N-(methyl-d₃)) carboxamide (5)

To dry DMF (100 mL) was added 4-aminophenol (9.54 g, 0.087 mol) and potassium tert-butoxide (10.3 g, 0.092 mol) in turn. The color of the solution turned into deep brown. After stirring at room temperature for 2 hours, to the reaction mixture was added 4-chloro-(N-methyl-d₃)pyridine-2-carboxamide (3) (13.68 g, 0.079 mol) and anhydrous potassium carbonate (6.5 g, 0.0467 mol), then warmed up to 80° C. and stirred over night. The reaction was completed by TLC detection. The reaction mixture was cooled to room temperature, and poured into a mixed solution of ethyl acetate (150 mL) and saturated brine (150 mL). The mixture was stirred and then stood for layering. The aqueous phase was extracted with ethyl acetate (3×100 mL). The extracted layers were combined, washed with saturated brine (3×100 mL) prior to drying over anhydrous sodium sulfate, and concentrated to afford 4-(4-aminophenoxy)-2-pyridine-(N-(methyl-d₃))carboxamide (18.00 g, 92% yield) which was light yellow.

¹H NMR (CDCl₃, 300 MHz): 8.32 (d, 1H), 7.99 (br, 1H), 7.66 (s, 1H), 6.91-6.85 (m, 3H), 6.69 (m, 2H), 3.70 (br, s, 2H).

3. Preparation of N-(4-chloro-3-(trifluoromethyl)phenyl)-N′-(4-(2-(N-(methyl-d₃)aminoformyl)-4-pyridyloxy)phenyl)urea (CM4307)

To methylene chloride (120 mL) was added 4-chloro-3-trifluoromethyl-phenylamine (15.39 g, 78.69 mmol) and N,N-carbonyldiimidazole (13.55 g, 83.6 mmol). After stirred at room temperature for 16 hours, to the reaction mixture was slowly added dropwise a solution of 4-(4-aminophenoxy)-2-pyridine-(N-(methyl-d₃))-carboxamide (18 g, 73 mmol) in methylene chloride (180 mL) and stirred at room temperature for another 18 hours. The reaction was completed by TLC detection. The mixture was concentrated to about 100 mL by removing methylene chloride through a rotary evaporator and stood for several hours at room temperature. A great amount of white solid precipitate formed. The solid was filtered and washed with abundant methylene chloride. The filtrate was concentrated by removing some solvent, and some solid precipitate formed again. Two parts of solid were combined and washed with abundant methylene chloride to afford N-(4-chloro-3-(trifluoromethyl)phenyl)-N′-(4-(2-(N-(methyl-d₃)aminoformyl)-4-pyridyloxy)phenyl)urea (CM4307, 20.04 g, 58% yield) as a white powder (pure product).

¹H NMR (CD₃OD, 300 MHz): 8.48 (d, 1H), 8.00 (d, 1H), 7.55 (m, 5H), 7.12 (d, 7.08 (s, 2H), ESI-HRMS m/z: C₂₁H₁₃D₃ClF₃N₄O₃, Calcd. 467.11, Found 490.07 (M+Na)⁺.

Furthermore, Compound CM4307 was dissolved in methylene chloride and reacted with benzoperoxoic acid to afford the oxidized derivative: 4-(4-(3-(4-chloro-3-(trifluoromethyl)phenyl)ureido)phenoxy)-2-(N-(methyl-d₃)aminoformyl)pyridine-1-oxide.

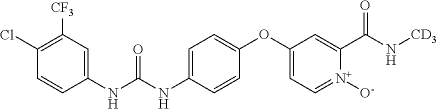 [SMILES: [2H]C([2H])([2H])NC(=O)c1cc(Oc2ccc(NC(=O)Nc3ccc(Cl)c(C(F)(F)F)c3)cc2)cc[n+]1[O-]]

### EXAMPLE 2

### Preparation of 4-chloropyridine-(N-(methyl-d₃))-2-carboxamide (3)

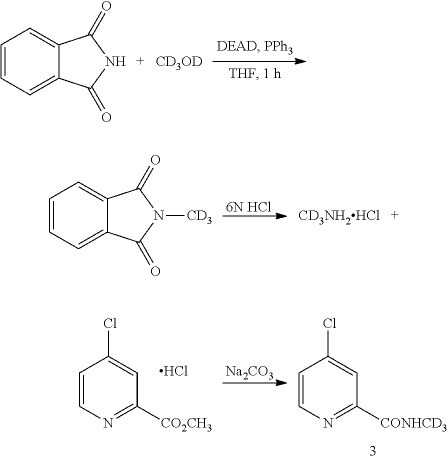

a) To a solution of phthalimide (14.7 g, 0.1 mol), deuterated methanol (3.78 g, 0.105 mol, 1.05 eq) and triphenylphosphine (28.8 g, 0.11 mol, 1.1 eq) in anhydrous tetrahydrofuran, a solution of DEAD (1.1 eq) in tetrahydrofuran was dropwise added under ice-bath condition. After addition, the mixture was stirred for 1 hour at room temperature. The mixture was purified by chromatography column, or the solvent in the mixture was removed, and then to the residue was added an appropriate amount of DCM and cooled in the refrigerator to precipitate the solid. The mixture was filtered and the filtrate was concentrated by rotary evaporator, and then the residue was purified by flash chromatography column to afford the pure product of 2-(N-(methyl-d₃))-isoindole-1,3-dione (14.8 g, 90% yield).

b) 2-(N-(methyl-d₃))-isoindole-1,3-dione (12.5 g, 0.077 mol) was dissolved in suitable amount of hydrochloric acid (6 N, 50 mL) and the mixture was refluxed for 24-30 hours in a sealed tube. The reaction mixture was cooled to room temperature and then cooled below 0° C. in the refrigerator to precipitate the solid. The solid was filtrated and washed with cold deionized water. The filtrate was collected, concentrated by rotary evaporator to remove water and dried to afford (methyl-d₃)amine hydrochloride salt. Anhydrous DCM (100 mL) was added in (methyl-d₃)amine hydrochloride salt and 4-chloro-pyridine-2-carboxylic acid methyl ester hydrochloride salt (6.52 g, 0.038 mol, 0.5 eq) and sodium carbonate (12.2 g, 0.12 mol, 1.5 eq) were added. The reaction flask was sealed and placed in the refrigerator for one day. After the reaction was completed by TLC detection, the reaction mixture was washed with water, dried, concentrated and purified by chromatography column to afford 4-chloro-pyridine-2-(N-(methyl-d₃))formamide (compound (3), 5.67 g, 86% yield). The structural feature was the same as Example 1.

### EXAMPLE 3

### Preparation of N-(4-ehloro-3-(trifluoromethyl)phenyl)-N′-(4-(2-(N-(methyl-d₂)aminoformyl)-4-pyridyloxy)phenyl)urea

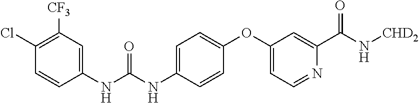

The title compound was prepared according to the method of Example 1, except that CD₃NH₂ was replaced by CD₂HNH₂.

### EXAMPLE 4

### Preparation of N-(4-chloro-3-(trilluoromethyl)phenyl)-N′-(4-(2-(N-(methyl-d)aminoformyl)-4-pyridyloxy)phenyl)urea

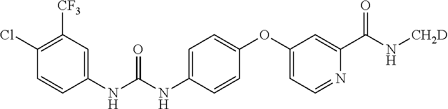

The title compound was prepared according to the method of Example 1, except that CD₃NH₂ was replaced by CDH₂NH₂.

### EXAMPLE 5

### Preparation of N-(4-chloro-3-(methyl-d₃)phenyl)-N′-(4-(2-(N-methylaminoformyl)-4-pyridyloxy)phenyl)urea

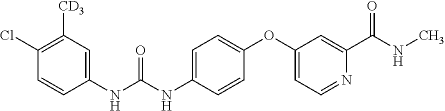 [SMILES: [2H]C([2H])([2H])c1cc(NC(=O)Nc2ccc(Oc3ccnc(C(=O)NC)c3)cc2)ccc1Cl]

The title compound was prepared according to the method of Example 1, except that 5-amino-2-chloro-methylbenzene was replaced by 5-amino-2-chloro-(methyl-d₃)benzene and CD₃NH₂ was replaced by CH₃NH₂.

### EXAMPLE 6

### Preparation of N-(4-chloro-3-(trifluoromethyl)phenyl)-N′-(4-(2-d-6-(N-methylaminoformyl)-4-pyridyloxy)phenyl)urea

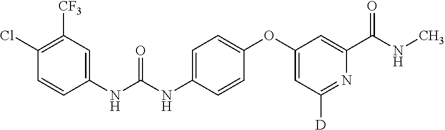 [SMILES: [2H]c1cc(Oc2ccc(NC(=O)Nc3ccc(Cl)c(C(F)(F)F)c3)cc2)cc(C(=O)NC)n1]

The title compound was prepared according to the method of Example 1, except that nicotinic acid was replaced by 2-d-6-carboxyl pyridine and CD₃NH₂ was replaced by CH₃NH₂.

### EXAMPLE 7

### Preparation of N-(4-chloro-3-(trifluoromethyl)phenyl)-N′-(2-d-4-(2-d-6-(N-methylaminoformyl)-4-pyridyloxy)phenyl)urea

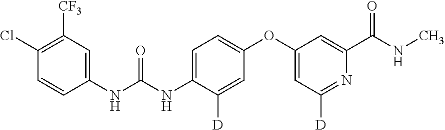 [SMILES: [2H]c1cc(Oc2ccc(NC(=O)Nc3ccc(Cl)c(C(F)(F)F)c3)c([2H])c2)cc(C(=O)NC)n1]

The title compound was prepared according to the method of Example 1, except that nicotinic acid was replaced by 2-d-6-carboxyl pyridine, 4-aminophenol was replaced by 3-d-4-aminophenol and CD₃NH₂ was replaced by CH₃NH₂.

### EXAMPLE 8

### Preparation of N-(4-chloro-3-(trifluoromethyl)phenyl)-N′-(2,6-d₂-4-(2-d-6-(N-methylaminoformyl)-4-pyridyloxy)phenyl)urea

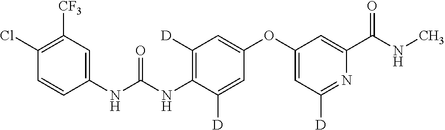 [SMILES: [2H]c1cc(Oc2cc([2H])c(NC(=O)Nc3ccc(Cl)c(C(F)(F)F)c3)c([2H])c2)cc(C(=O)NC)n1]

The title compound was prepared according to the method of Example 1, except that nicotinic acid was replaced by 2-d-6-carboxyl pyridine, 4-aminophenol was replaced by 3,5-d₂-4-aminophenol and CD₃NH₂ was replaced by CH₃NH₂.

### EXAMPLE 9

### Preparation of N-(4-chloro-3-(methyl-d₃)phenyl)-N′-(4-(2-d-6-(N-methylaminoformyl)-4-pyridyloxy)phenyl)urea

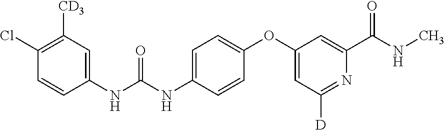 [SMILES: [2H]c1cc(Oc2ccc(NC(=O)Nc3ccc(Cl)c(C([2H])([2H])[2H])c3)cc2)cc(C(=O)NC)n1]

The title compound was prepared according to the method of Example 1, except that nicotinic acid was replaced by 2-d-6-carboxyl pyridine, 5-amino-2-chloro-trifluorobenzene was replaced by 5-amino-2-chloro-(methyl-d₃)benzene and CD₃NH₂ was replaced by CH₃NH₂.

### EXAMPLE 10

### Preparation of N-(4-chloro-3-(trifluoromethyl)phenyl)-N′-(4-(2-(N-methyl-(methyl-d₃)aminoformyl)-4-pyridyloxy)phenyl)urea

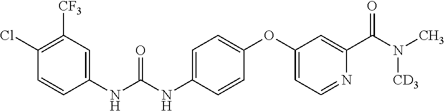 [SMILES: [2H]C([2H])([2H])N(C)C(=O)c1cc(Oc2ccc(NC(=O)Nc3ccc(Cl)c(C(F)(F)F)c3)cc2)ccn1]

The title compound was prepared according to the method of Example 1, except that CD₃NH₂ was replaced by CD₃N(CH₃)H.

### EXAMPLE 11

### Preparation of N-(4-chloro-3-(trifluoromethyl)phenyl)-N′-(4-(2-(N,N-di(methyl-d₃)aminoformyl)-4-pyridyloxy)phenyl)urea

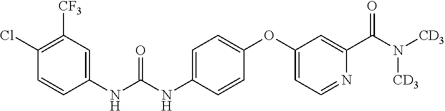 [SMILES: [2H]C([2H])([2H])N(C(=O)c1cc(Oc2ccc(NC(=O)Nc3ccc(Cl)c(C(F)(F)F)c3)cc2)ccn1)C([2H])([2H])[2H]]

The title compound was prepared according to the method of Example 1, except that CD₃NH₂ was replaced by (CD₃)₂NH.

### EXAMPLE 12

### Preparation of N-(4-chloro-3-(trifluoromethyl)phenyl)-N′-(2,6-d₂-4-(2-(N-(methyl-d₃)aminoformyl)-4-pyridyloxy)phenyl)urea

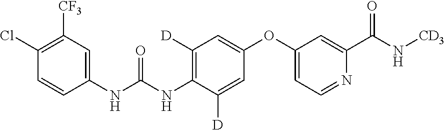 [SMILES: [2H]c1cc(Oc2ccnc(C(=O)NC([2H])([2H])[2H])c2)cc([2H])c1NC(=O)Nc1ccc(Cl)c(C(F)(F)F)c1]

The title compound was prepared according to the method of Example 1, except that 4-aminophenol was replaced by 3,5-d₂-4-aminophenol.

### EXAMPLE 13

### Preparation of N-(4-chloro-3-(trifluoromethyl)phenyl)-N′-(4-(2-d-6-(N-(methyl-d₃)aminoformyl)-4-pyridyloxy)phenyl)urea

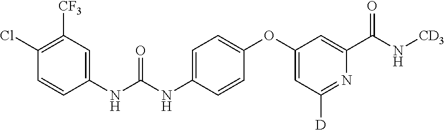 [SMILES: [2H]c1cc(Oc2ccc(NC(=O)Nc3ccc(Cl)c(C(F)(F)F)c3)cc2)cc(C(=O)NC([2H])([2H])[2H])n1]

The title compound was prepared according to the method of Example 1, except that nicotinic acid was replaced by 2-d-6-carboxyl pyridine.

### EXAMPLE 14

### Preparation of N-(4-chloro-3-(trifluoromethyl)phenyl)-N′-(4-(2-(N-(ethyl-1′,1′-d₂)aminoformyl)-4-pyridyloxy)phenyl)urea

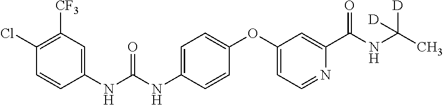 [SMILES: [2H]C([2H])(C)NC(=O)c1cc(Oc2ccc(NC(=O)Nc3ccc(Cl)c(C(F)(F)F)c3)cc2)ccn1]

The title compound was prepared according to the method of Example 1, except that CD₃NH₂ was replaced by CH₃CD₂NH₂.

### EXAMPLE 15

### Preparation of N-(4-chloro-3-(trifluoromethyl)phenyl)-N′-(4-(2-(N-(ethyl-d₅)aminoformyl)-4-pyridyloxy)phenyl)urea

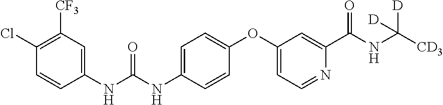 [SMILES: [2H]C([2H])([2H])C([2H])([2H])NC(=O)c1cc(Oc2ccc(NC(=O)Nc3ccc(Cl)c(C(F)(F)F)c3)cc2)ccn1]

The title compound was prepared according to the method of Example 1, except that CD₃NH₂ was replaced by CD₃CD₂NH₂.

### EXAMPLE 16

### Preparation of N-(4-chloro-3-(methyl-d₃)phenyl)-N′-(4-(2-(N-(methyl-d₃)aminoformyl)-4-pyridyloxy)phenyl)urea

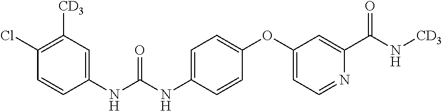 [SMILES: [2H]C([2H])([2H])NC(=O)c1cc(Oc2ccc(NC(=O)Nc3ccc(Cl)c(C([2H])([2H])[2H])c3)cc2)ccn1]

The title compound was prepared according to the method of Example 1, except that 5-amino-2-chloro-trifluoromethylbenzene was replaced by 5-amino-2-chloro-(methyl-d₃)benzene.

### EXAMPLE 17

### Pharmacokinetic Evaluation in Rats

8 male Sprague-Dawley rats (7-8 weeks old and body weight about 210 g) were divided into two groups, 4 in each group (rat No.: control group was 13-16; experimental group was 9-12), and orally administrated a single dose at 3 mg/kg of either compound: (a) the undeuterated N-(4-chloro-3-(trifluoromethyl)phenyl)-N′-(4-(2-(N-methyl-aminoformyl)-4-pyridyloxy)phenyl)urea (control compound CM4306) or (b) N-(4-chloro-3-(trifluoromethyl)phenyl)-N′-(4-(2-(N-(methyl-d₃)-aminoformyl)-4-pyridyloxy)phenyl)urea (Compound CM4307 of the invention) prepared in Example 1. The pharmacokinetics differences were compared.

The rats were fed with the standard feed, given water and chlordiazepoxide. Chlordiazepoxide was stopped at the last night before experiment, and given again two hours after the administration of the compound. The rats were fasted for 16 hours before the test. The compound was dissolved in 30% PEG400. The time for collecting orbital blood was 0.083, 0.25, 0.5, 1, 2, 4, 6, 8 and 24 hours after administrating the compounds.

The rats were anaesthetized briefly by inhaling ether. A 300 μL orbital blood sample was collected into the tubes containing a 30 μl 1% heparin saline solution. The tubes were dried overnight at 60° C. before being used. After the blood samples were sequentially collected, the rats were anaesthetized by ether and sacrificed.

After the blood samples were collected, the tubes were gently reversed at least five times immediately to mix the contents sufficiently, and placed on the ice. The blood samples were centrifuged at 4° C. at 5000 rpm for 5 minutes to separate the serum and red blood cells. 100 μL serum was removed to a clean plastic centrifugal tube by pipettor, and the name of the compound and time point were labeled on the tube. Serum was stored at −80° C. before LC-MS analysis.

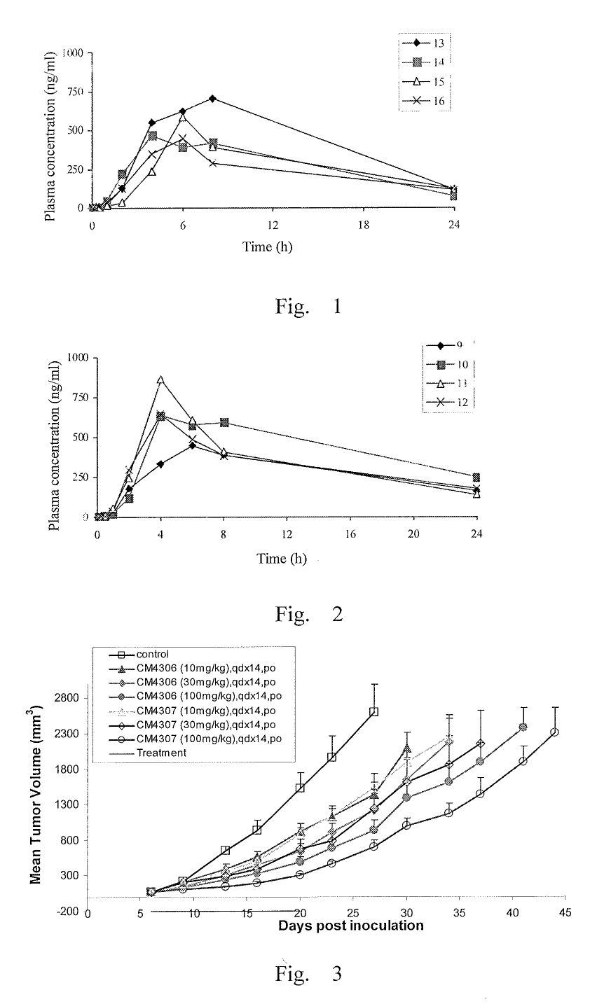

The results were shown in FIGS. 1-2. The results showed that, compared with CM4306, the half-life (T_{1/2}) of CM4307 was longer [11.3±2.1 hours for CM4307 and 8.6±1.4 hours for CM4306, respectively], area under the curve (AUC_{0-∞}) of CM4307 was significantly increased [11255±2472 ng·h/mL for CM4307 and 7328±336 ng·h/mL for CM4306, respectively], and apparent clearance of CM4307 was reduced [275±52 mL/h/kg for CM4307 and 410±18.7 mL/h/kg for CM4306, respectively].

The above results showed that, the compound of the present invention had better pharmacokinetics properties in the animal, and thus had better pharmacodynamics and therapeutic effects.

In addition, the metabolism for the compound of the present invention in organism was changed through deuteration. In particular, the hydroxylation of phenyl became more difficult, which led to the reduction of first-pass effect. In such cases, the dose can be changed, long-acting preparations can be formed, and the applicability can be improved by using long-acting preparations.

Furthermore, the pharmacokinetics was also changed through deuteration. Since another hydrate film is frilly formed by deuterated compounds, the distribution of deuterated compounds in organisms is significantly different from that of the non-deuterated compounds.

### EXAMPLE 18

### The Pharmacodynamic Evaluation of CM4307 for Inhibiting Tumor Growth of Human Hepatocellular Carcinoma SMMC-7721 in Nude Mice Xenograft Model

70 Balb/c nu/nu nude mice, 6 weeks old, female, were bought from Shanghai Experimental Animal Resource Center (Shanghai B&K Universal Group Limited).

SMMC-7721 cells were commercially available from Shanghai Institutes for Biological Science, CAS (Shanghai, China).

The establishment of tumor nude mice xenograft model: SMMC-7721 cells in logarithmic growth period were cultured. After cell number was counted, the cells were suspended in 1×PBS, and the number of the cell in suspension was adjusted to 1.5×10⁷/ml. The tumor cells were inoculated under the skin of right armpit of nude mice with a 1 ml syringe, 3×10⁶/0.2 ml/mice. 70 nude mice were inoculated in total.

When the tumor size reached 30-130 mm³, 58 mice were divided randomly into different groups. The difference of the mean value of tumor volume in each group was less than 10%, and drugs were started to be administrated.

The test doses for each group were listed in the following table.

| | Ani- | | Adminis- | Dose | |
|---|---|---|---|---|---|
| Group | mal | Compounds | tration | (mg/kg) | Method |
| 1 | 10 | control | po | 0.1 ml/10 g | qd × 2 weeks |
| | | (solvent) | | BW | |
| 2 | 8 | CM4306 | po | 10 mg/kg | qd × 2 weeks |
| 3 | 8 | CM4306 | po | 30 mg/kg | qd × 2 weeks |
| 4 | 8 | CM4306 | po | 100 mg/kg | qd × 2 weeks |
| 5 | 8 | CM4307 | po | 10 mg/kg | qd × 2 weeks |
| 6 | 8 | CM4307 | po | 30 mg/kg | qd × 2 weeks |
| 7 | 8 | CM4307 | po | 100 mg/kg | qd × 2 weeks |

Animal body weight and tumor size were tested twice a week during the experiment. Clinical symptoms were recorded every day. At the end of the administration, the tumor size was recorded by taking pictures. One mouse was sacrificed in each group and tumor tissue was taken and fixed in 4% paraformaldehyde. Observation was continued after the administration, and when the mean size of tumor was larger than 2000 mm³, or the dying status appeared, the animals were sacrificed, gross anatomy was conducted, and the tumor tissue was taken and fixed in 4% paraformaldehyde.

The formula for calculating the tumor volume (TV) is: TV=a×b²/2, wherein a, b independently represent the length and the breadth of the tumor. The formula for calculating the relative tumor volume (RTV) is: RTV=Vt/V₀, wherein V₀ is the tumor volume at the beginning of the administration, and Vt is the tumor weight when measured. The index for evaluating the antitumor activity is relative tumor increment rate T/C (%), and the formula is: T/C (%)=(T_{RTV}/C_{RTV})×100%, wherein, T_{RTV} is the RTV of the treatment group, and C_{RTV} is the RTV of the negative control group.

Evaluation standard for efficacy: it is effective if the relative tumor increment rate T/C (%) is <40% and p<0.05 by statistics analysis.

The results were shown in FIG. 3. CM4306 and CM4307 were intragastric administrated every day for 2 weeks at doses of 10, 30, 100 mg/kg respectively, and both compounds showed the dose-dependent effect of the inhibition of tumor growth. At the end of administration, T/C % of CM4306 was 56.9%, 40.6% and 32.2%, respectively. T/C % of CM4307 was 53.6%, 40.8% and 19.6%. T/C % for 100 mg/kg dose groups was <40%, and tumor volume was significantly different (p<0.01) from the control group, indicating the significant effect in inhibiting tumor growth.

Compared with CM4306, the inhibitory efficacy of tumor growth at dosing 100 mg/kg of CM4307 was stronger (the T/C % for CM4307 and CM4306 is 19.6% and 32.2%, respectively, at day 15), there was significant difference in tumor volume between groups (p<0.01). Compared with CM4306, the absolute value of tumor inhibition rate for CM4307 increased more than 10%, the relative value increased about 60% (32.2%/19.6%−1=64%), and CM4307 showed more significant effect for inhibiting tumor growth.

In addition, during the experiment, no other drug-relevant toxic effects were observed.

### EXAMPLE 19

### Pharmaceutical Compositions

| | |
|---|---|
| Compound CM4307 (Example 1) | 20 g |
| Starch | 140 g |
| Microcrystalline cellulose | 60 g |

By routine methods, these substances were blended evenly, and loaded into ordinary gelatin capsules, thereby forming 1000 capsules.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1.-14. (canceled)

15. A purified or isolated deuterated w-diphenylurea compound or a crystal form, pharmaceutically acceptable salt, hydrate or solvate thereof, wherein the compound is N-(4-chloro-3-(trifluoromethyl)phenyl)-N′-(4-(2-(N-(methyl-d₃)aminoformyl)-4-pyridyloxy)phenyl)urea:

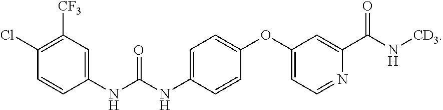 [SMILES: [2H]C([2H])([2H])NC(=O)c1cc(Oc2ccc(NC(=O)Nc3ccc(Cl)c(C(F)(F)F)c3)cc2)ccn1]

16. The compound of claim 15, wherein the deuterium content in the deuterium-substituted position is >95%.

17. The compound of claim 15, wherein each of H, N, O, C, F and Cl atoms in the compound is present at its natural abundance except the D in the deuterium-substituted position.

18. A method for preparing a pharmaceutical composition comprising: mixing a pharmaceutically acceptable carrier with the compound according to claim 15, or the crystal form, pharmaceutically acceptable salt, hydrate or solvate thereof, thereby forming the pharmaceutical composition.

19. A pharmaceutical composition comprising a pharmaceutically acceptable carrier and the compound according to claim 15, or the crystal form, pharmaceutically acceptable salt, hydrate or solvate thereof.

20. The pharmaceutical composition according to claim 19, wherein the pharmaceutical composition further comprises an additional medicament for treating cancer, cardiovascular diseases, inflammation, immunological diseases, nephrosis, angiogenesis, or prostatosis.

21. A method of inhibiting a phosphokinase in a subject in need of, comprising administering to the subject the pharmaceutical composition of claim 19.

22. The method of claim 21, wherein the phosphokinase is a raf kinase.

23. A method of treating a disease in a subject in need of, comprising administrating to the subject the pharmaceutical composition of claim 19.

24. The method of claim 23, wherein the disease is selected from the group consisting of cancers, cardiovascular diseases, inflammation, immune diseases, nephrosis, angiogenesis, and prostatosis, provided that the cancer is not kidney cancer.

25. The method of claim 24, wherein the cancer is selected from the group consisting of non-small-cell lung cancer, uterine cancer, rectal cancer, cerebral cancer, head cancer, neck cancer, bladder cancer, prostate cancer, breast cancer, solid tumor, leukaemia, liver cancer, gastric cancer, colorectal cancer, and pancreatic cancer.

26. The method of claim 23, wherein the daily dose of the compound is 20-500 mg for a person weighed 60 kg.

27. The method of claim 26, wherein the daily dose of the compound is 20-500 mg for a person weighed 60 kg.

28. A method for preparing a compound of N-(4-chloro-3-(trifluoromethyl)phenyl)-N-(4-(2-(N-(methyl-d₃)aminoformyl)-4-pyridyloxy)phenyl)urea: 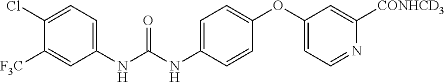
comprising a step selected from the group consisting of:
(a) in an inert solvent and in the presence of a base, reacting compound III with compound V to form the compound, wherein X is Cl, Br, or I: 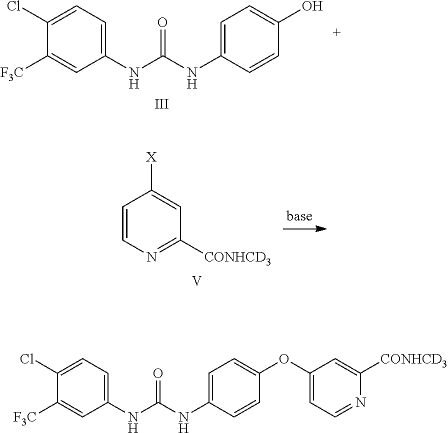
(b) in an inert solvent, reacting compound IX with CD₃NH₂ or CD₃NH₂.HCl to form the compound, wherein R is C1-C8 straight-chain or branched chain alkyl or aryl: 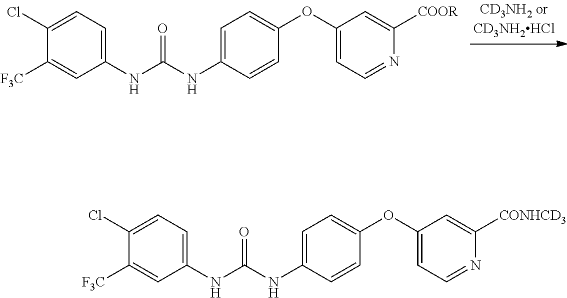
(c) in an inert solvent, reacting 1-chloro-4-isocyanato-2-(trifluoromethyl)benzene (VIII) with compound 5 to form the compound: 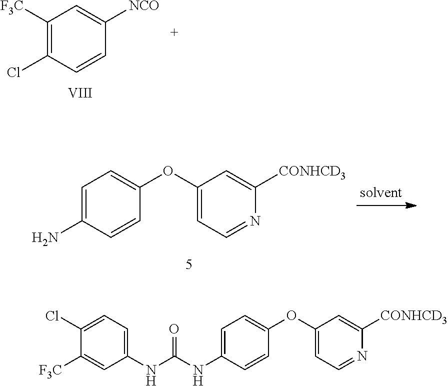
and (d) in an inert solvent and in the presence of CDI and CH₂Cl₂, reacting compound 5 with compound 6 to form the compound:

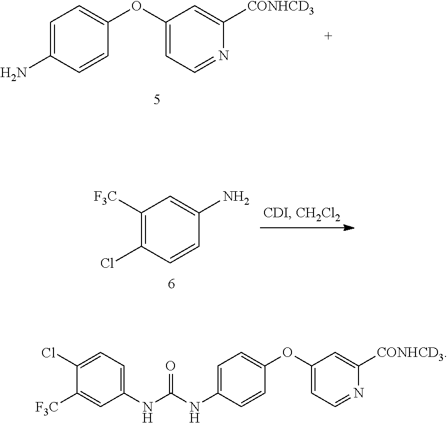

29. An intermediate having Formula (A), (B) or (C):

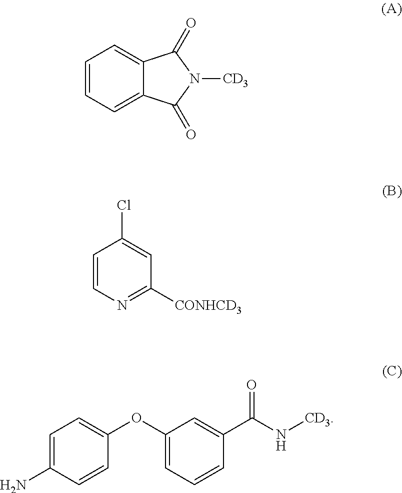

30. A method for preparing a deuterated ω-diphenylurea compound comprising using one or more of the intermediates of claim 29.
